(19) United States
(12) Patent Application Publication
(10) Pub. No.: US 2004/0122447 A1
(43) Pub. Date: Jun. 24, 2004

(54) Brush tip for use with a hand-held debridement device

(76) Inventors: Kim R. Harmon, Mineral City, OH (US); Tim A. Donaldson, Massillon, OH (US); Jeffrey B. Boggs, Peachtree City, GA (US)

Correspondence Address: BAKER & DANIELS, 111 E. WAYNE STREET, SUITE 800, FORT WAYNE, IN, 46802

(21) Appl. No.: 10/325,537

(22) Filed: Dec. 19, 2002

(51) Int. Cl. A61B 17/50

(52) U.S. Cl. 606/131

(57) ABSTRACT

A brush debridement tip for use with hand-held wound debridement devices. The brush debridement tip of the present invention provides concurrent irrigation and suction to the area to be debrided and includes a plurality of bristles extending therefrom. The plurality of bristles have varying lengths to advantageously allow for mechanical debridement of, e.g., resected or otherwise formed having irregular shapes. The brush debridement tip further includes an elongate aperture allowing passage of a fan spray of irrigation fluid therethrough.

### BACKGROUND OF THE INVENTION

1. Field of the Invention

This invention relates generally to hand-held wound debridement devices, and more particularly, to a brush tip for such devices.

2. Description of the Related Art

Debridement is the surgical excision of dead, devitalized, and contaminated tissue, and/or the removal of foreign matter from a wound. Debridement is advantageous because it cleanses the wound and lowers the bacterial count of wound tissues, thereby facilitating the healing process. Hand-held wound debridement devices generally include an irrigation system to provide pressurized streams of fluid to dislodge unwanted tissue and foreign matter from a wound and a suction system to collect the dislodged matter and irrigation fluids. Hand-held wound debridement systems which utilize pressurized streams of fluid to dislodge unwanted tissue and foreign matter from a wound generally provide an efficient and effective means of debriding a wound. Debridement devices are further utilized to clean bones which have undergone shaping prior to positioning an implant in the shaped bone. When cleaning shaped bones, it can prove advantageous to utilize a debridement tip capable of mechanically scrubbing the shaped bone while applying irrigation fluid and suction thereto.

What is needed in the art is a debridement tip which effectively provides concurrent irrigation and suction to the area to be debrided, and which, at the same time, provides surface scrubbing action.

Also needed in the art is a debridement tip accommodating mechanical scrubbing and which is capable of emitting a fan-shaped spray pattern of irrigation fluid.

Also needed in the art is a debridement tip capable of effectively mechanically scubbing a resected or otherwise shaped bone having an irregular surface to be debrided.

### SUMMARY OF THE INVENTION

The present invention is a brush debridement tip for use with hand-held debridement devices, such as the various Zimmer Pulsavac® hand-held debridement devices. The brush debridement tip of the present invention includes a proximal adapter for engaging a mating portion of a hand-held debridement device and a distal bristled tip for scrubbing contact with the bone being cleaned. Advantageously, the debridement tip of the present invention can be a single use, disposable item, and may be easily attached and detached from the debridement device.

In one form of the present invention the distal, bristled end of the debridement tip comprises a substantially circular end having a plurality of substantially transverse bristles extending therefrom. Advantageously, the bristles vary in length, to accommodate scrubbing the irregular surfaces of the bone being cleaned. The distal end of the brush debridement tip of the present invention further includes a single opening for delivering concurrent irrigation and suction to the area to be debrided. The opening in the distal end of the brush debridement tip of the present invention is shaped to allow for delivery of irrigation fluid in a fan-shaped spray pattern, allowing for maximum cleaning efficiency.

The brush debridement tip of the present invention is advantageous for numerous reasons. For instance, the variable length bristles of the brush debridement tip allows for effective scrubbing of irregular surfaces. The improved scrubbing action of the brush debridement tip of the present invention advantageously yields more efficient debridement relative to a brush having bristles of consistent length. Moreover, the fan-shaped spray pattern made possible by the shape of the opening in the distal end of the brush debridement tip of the present invention advantageously improves cleaning efficiency of the brush debridement tip of the present invention.

The invention, in one form thereof, comprises a debridement tip having a proximal end and a distal end, with the proximal end being adapted for connection to an irrigation source and a suction source. The debridement tip of this form of the present invention includes a fitting adjacent the proximal end, with the fitting including an irrigation port and a suction port for connecting the debridement tip to the irrigation source and the suction source, respectively. The debridement tip further includes an irrigation passage connected in fluid communication with the irrigation port and operable to convey a quantity of irrigation fluid from the irrigation port to the distal end of the debridement tip as well as a suction passage connected in fluid communication with the suction port and operable to convey a quantity of suction from the suction port to the distal end of the debridement tip. Finally, the debridement tip of this form of the present invention includes a plurality of bristles extending distally from the distal end of the debridement tip, with a first of the plurality of bristles being a distance longer than the second plurality of bristles. In one exemplary embodiment, the longer bristles measures 1-3 mm longer than the shorter bristles.

The invention, in another form thereof, comprises a debridement tip having a proximal end and a distal end and including delivery means for delivering a quantity of irrigation fluid and a quantity of suction to an area to be debrided as well as bristle contact means for contacting an irregularly shaped surface.

The invention, in a further form thereof comprises a debridement tip having a proximal end and a distal end, with the proximal end being adapted for connection to an irrigation source and a suction source. The debridement tip of this form of the present invention includes a fitting adjacent the proximal end, with the fitting including an irrigation port and a suction port for connecting the debridement tip to the irrigation source and the suction source, respectively. The debridement tip of this form of the present invention further includes an irrigation passage connected in fluid communication with the irrigation port, with the irrigation passage operable to convey a quantity of irrigation fluid from the irrigation port to the distal end of the debridement tip. The debridement tip further includes a suction passage connected in fluid communication with the suction port, with the suction passage operable to convey a quantity of suction from the suction port to the distal end of the debridement tip. Finally, the debridement tip of this form of the present invention includes a fan spray tip, with the irrigation passage terminating in the fan spray tip whereby the quantity of irrigation fluid traversing the irrigation passage is ejected from the fan spray tip in a fan spray pattern. The distal end of the debridement tip further includes an elongate opening through which the irrigation fluid may be ejected in a fan spray pattern.

An object of the present invention is to provide a brush debridement tip which effectively provides concurrent irrigation and suction to the area to be debrided, and which, at the same time, provides surface scrubbing action.

Yet another object of the present invention is to provide a brush tip debridement tip capable of emitting a fan-shaped spray pattern.

### BRIEF DESCRIPTION OF THE DRAWINGS

The aforementioned and other features and objects of this invention, and the manner of attaining them, will become apparent and the invention itself will be better understood by reference to the following description of an embodiment of the invention taken in conjunction with the accompanying drawings, wherein:

FIG. 1 is a perspective view of a hand-held wound debridement device equipped with the brush debridement tip of the present invention; and

FIG. 2 is a cross-sectional view of the brush debridement tip shown in FIG. 1; and

FIG. 3 is a distal plan view of the brush debridement tip shown in FIGS. 1 and 2.

Corresponding reference characters indicate corresponding parts throughout the several views. Although the drawings represent an embodiment of the present invention, the drawings are not necessarily to scale and certain features may be exaggerated in order to better illustrate and explain the present invention. The exemplification set out herein illustrates an embodiment of the invention, in one form, and such exemplification is not to be construed as limiting the scope of the invention in any manner.

### DETAILED DESCRIPTION OF THE PRESENT INVENTION

The embodiment disclosed below is not intended to be exhaustive or to limit the invention to the precise form disclosed in the detailed description. Rather, the embodiment is chosen and described so that others skilled in the art might utilize its teachings.

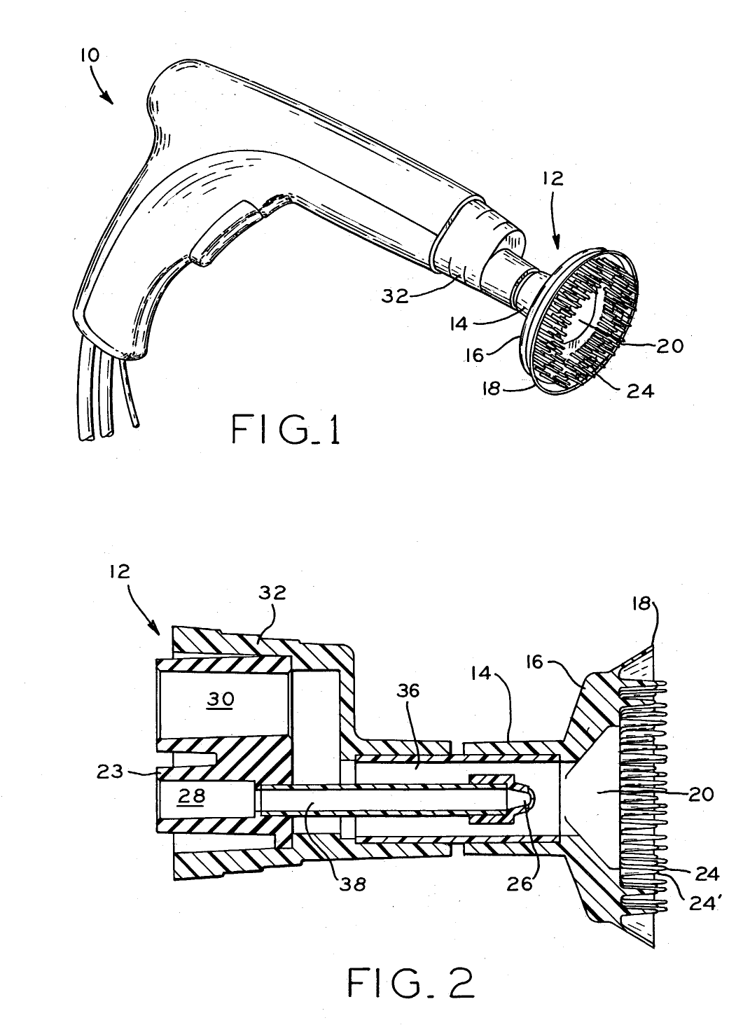

With reference to FIGS. 1 and 2, hand-held wound debridement device 10 is equipped with brush debridement tip 12 of the present invention. Brush debridement tip 12 includes fitting 32 adapted to couple brush debridement tip 12 to hand-held wound debridement device 10. Brush debridement tip 12 is designed for single use, and fitting 32 may be easily attached to and detached from hand-held wound debridement device 10, as necessary.

Fitting 32 includes fluid port 28 and suction port 30 adapted to couple brush debridement tip 12 to a source of irrigation fluid and suction, respectively. Fluid port 28 and suction port 30 are part of unitary structure 23, which is connected to tip adapter housing 32 via, e.g., sonic welds. Suction port 30 is in fluid communication with suction passage 36 (discussed hereinafter), and is independent from fluid port 28.

Fluid port 28 is connected to fluid passage 38 which is fitted with spray tip 26. Fluid passage 38, in conjunction with spray tip 26, is adapted to provide the desired supply of irrigation fluid to the area to be debrided. Spray tip 26, as illustrated in FIG. 2, is adapted to emit a fan-shaped spray pattern of irrigation fluid.

Fluid passage 38 extends from fluid port 28 through suction passage 36. Suction passage 36 and fluid passage 38 are independent of one another, and provide concurrent irrigation and suction to the area being debrided. Suction passage 36 extends radially as neck region 14 of brush debridement tip 12 transitions into radially expanding region 16. As illustrated in FIG. 2, suction passage 36 extends into distal open end 20 of brush debridement tip 12.

Radially expanding region 16 includes a plurality of bristles 24, 24′ extending distally therefrom as illustrated in FIG. 2. As illustrated in FIG. 2, the bristles extending from radially expanding region 16 have varying lengths, with bristles 24 being longer than bristles 24′. In one exemplary embodiment, bristles 24 are 1-3 mm longer than bristles 24′. In a further exemplary embodiment, bristles 24 are about 1 mm longer than bristles 24′. While illustrated as utilizing two different bristle lengths, the brush debridement tip of the present invention may employ three or more bristle lengths. Utilizing bristles of varying lengths allows the debridement tip of the present invention to effectively scrub an irregular surface, i.e., a non-flat surface. In the case of an irregular surface, shorter bristles 24′ may contact protruding regions of the surface being scrubbed, while longer bristles 24 contact depressed regions of the surface being scrubbed. In this way, bristles 24, 24′ can contact an irregularly shaped surface and supply scrubbing action thereto.

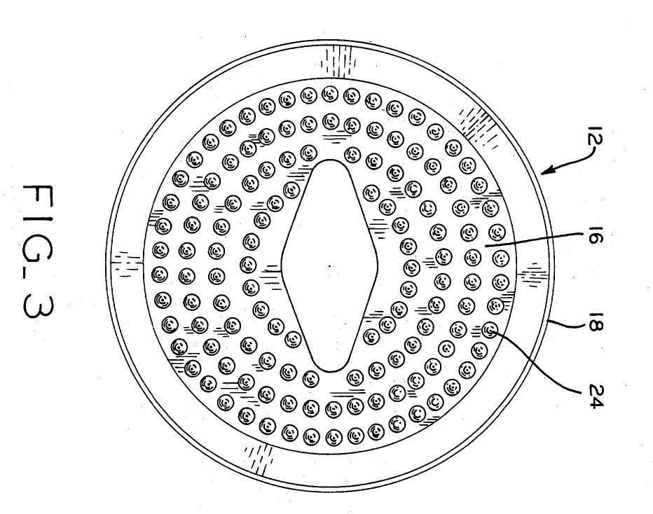

As illustrated in FIG. 3, distal open end 20 comprises an elongate opening accommodating a fan spray exiting therethrough. Distal open end 20 may be formed as a generally elongate aperture as depicted in FIG. 3 or may comprise a generally eliptical aperture.

In use, bristles 24, 24′ are rubbed against a resected or otherwise shaped bone while irrigation fluid and suction exits distal open end 20 of brush debridement tip 12. In this way, bristles 24 work together with irrigation fluid emitted from spray tip 26 to dislodge unwanted tissue and foreign matter from the resected bone, with the suction provided through suction passage 36 working to remove the dislodged material and dispensed irrigation fluid. Distal open end 20 is shaped to accommodate passage of a fan shaped spray of irrigation fluid therethrough while also accommodating passage of suction therethrough for the collection of dislodged material from the area being debrided as well as irrigation fluid.

Brush tip 12 terminates in rim 18 which extends from radially expanding region 16 and surrounds bristles 24, 24′. Rim 18 facilitates collection of the unwanted tissue and foreign matter dislodged from the area being debrided and shields the operator from irrigation fluid applied to the area being debrided.

While this invention has been described as having an exemplary design, the present invention may be further modified within the spirit and scope of the disclosure. This application is therefore intended to cover any variations, uses, or adaptations of the invention using its general principles. Further, this application is intended to cover such departures from the present disclosure as come within known or customary practice in the art to which this invention pertains.

What is claimed:

1. A debridement tip having a proximal end and a distal end, the proximal end being adapted for connection to an irrigation source and a suction source, the debridement tip comprising:
a fitting adjacent the proximal end, the fitting including an irrigation port and a suction port for connecting the debridement tip to the irrigation source and the suction source, respectively;
an irrigation passage connected in fluid communication with said irrigation port, said irrigation passage operable to convey a quantity of irrigation fluid from said irrigation port to the distal end of the debridement tip;
a suction passage connected in fluid communication with said suction port, said suction passage operable to convey a quantity of suction from said suction port to the distal end of the debridement tip;
and
a plurality of bristles extending distally from the distal end of the debridement tip, a first of said plurality of bristles a distance longer than a second of said plurality of bristles.

2. The debridement tip of claim 1, wherein the distal end of the debridement tip includes an elongate opening.

3. The debridement tip of claim 2, wherein said elongate opening comprises an elliptical opening.

4. The debridement tip of claim 1, wherein said distance measures 1-3 mm.

5. A debridement tip having a proximal end and a distal end, the debridement tip comprising:
delivery means for delivering a quantity of irrigation fluid and a quantity of suction to an area to be debrided; and
bristle contact means for contacting an irregularly shaped surface.

6. The debridement tip of claim 5, wherein said bristle contact means comprises a plurality of bristles extending distally from the distal end, a first of said plurality of bristles a distance longer than a second of said plurality of bristles.

7. The debridement tip of claim 6, wherein said distance measures 1-3 mm.

8. A debridement tip having a proximal end and a distal end, the proximal end being adapted for connection to an irrigation source and a suction source, the debridement tip comprising;
a fitting adjacent the proximal end, the fitting including an irrigation port and a suction port for connecting the debridement tip to the irrigation source and the suction source, respectively,
an irrigation passage connected in fluid communication with said irrigation port, said irrigation passage operable to convey a quantity of irrigation fluid from said irrigation port to the distal end of the debridement tip;
a suction passage connected in fluid communication with said suction port, said suction passage operable to convey a quantity of suction from said suction port to the distal end of the debridement tip; and
a fan spray tip, said irrigation passage terminating in said fan spray tip whereby said quantity of irrigation fluid is ejected from said fan spray tip in a fan spray pattern, and wherein the distal end of the debridement tip includes an elongate opening through which said irrigation fluid may be ejected in said fan spray pattern.

9. The debridement tip of claim 8, further comprising:
a plurality of bristles extending distally from the distal end of the debridement tip, a first of said plurality of bristles a distance longer than a second of said plurality of bristles.

10. The debridement tip of claim 9, wherein said distance measures 1-3 mm.
